(19) United States
(12) Patent Application Publication
(10) Pub. No.: US 2012/0103358 A1
(43) Pub. Date: May 3, 2012

(54) CAPILLARY COMPOSITION FOR TREATING HAIR

(75) Inventors: Samira Khenniche, Clichy (FR); Isabelle Rollat-Corvol, Paris (FR); Sophie Bourel, Suresnes (FR)

(73) Assignee: L'OREAL, Paris, FR (FR)

(21) Appl. No.: 13/262,415

(22) PCT Filed: Mar. 31, 2009
(86) PCT No.: PCT/FR2009/050550

§ 371 Date: Jan. 13, 2012

(51) Int. Cl. A61K 8/73; A61K 8/72; A61K 8/81; A61K 8/84; A61Q 5/10; A61Q 5/00; A61Q 5/12; A61Q 5/04; A61Q 5/08; A61K 8/02; A61K 8/87

(52) U.S. Cl. 132/204; 424/401; 424/70.11; 424/70.13; 424/70.16; 424/70.15; 424/70.17; 132/202; 132/208

(57) ABSTRACT

The invention relates to a capillary composition comprising at least particles of pumice, at least one cationic surfactant having at least one ester function, and at least one non-siliconised polymer.

The present invention relates to the treatment of human hair.

Hair may be impaired by chemical treatments such as dyeing or permanent waving, or by mechanical stresses such as disentangling or blow-drying. The mechanical, morphological and physicochemical properties of the surface of the hair, and especially of the cuticle, the outer layer of the hair with a scaly structure, are thereby modified. In particular, in the course of these treatments or stresses, the scales of the cuticle become raised and their edges, which are normally uniform, become jagged. These deteriorations may have several consequences. Firstly, the hair is less smooth and less easy to disentangle. Secondly, the active agents of a hair treatment product, for example a conditioner, are liable to be deposited non-uniformly on the hair. Now, it may prove desirable to deposit the care active agents uniformly over the hair as a whole.

It is known practice to care for damaged hair by applying thereto a care product comprising, for example, specific polymers such as silicones or polymers bearing cationic charges. However, the improvement of the hair thus treated is only temporary, since, once the care product has been removed, for example after one or more washes, the hair regains its original state.

There is a need for compositions, processes and kits that enable long-lasting treatment of the hair, in particular hair that is damaged at the surface.

The invention is directed, inter alia, toward satisfying this need, and it achieves this by means of a hair treatment process using a haircare composition comprising at least pumice particles, one or more cationic surfactants comprising at least one ester function and one or more nonsilicone polymers.

A first subject of the invention is thus a haircare composition comprising at least pumice particles, one or more cationic surfactants comprising at least one ester function and one or more nonsilicone polymers.

A second subject of the invention is a hair treatment process comprising at least the step consisting in placing the hair in contact with a haircare composition as defined above.

As emerges from the examples hereinbelow, the treatment of keratin fibers, and especially the hair, with a composition in accordance with the invention makes it possible to care for the keratin fiber homogeneously without impairing it, by unifying the deposition of the treating agents. Excellent smoothing of keratin fibers and controlled volume of the hairstyle are thus obtained. These effects are all the more noteworthy when the hair is sensitized and/or thick.

These effects are long-lasting and especially withstand shampoo washing.

The process may also include the step consisting in combing and/or rinsing the hair after said treatment.

The process may also advantageously be used for smoothing the hair.

The invention may also make it possible to prepare the hair for a hair post-treatment such as the application of a conditioner, a dye, a permanent-waving product, a hair-relaxing product, a bleaching product or the like.

The process may thus include the step consisting in subjecting the hair to a post-treatment, after treatment using the haircare composition of the invention, the post-treatment being chosen from the application of a conditioner, a permanent-waving product, a hair-relaxing product or a hair dyeing or bleaching product, this list not being limiting.

The hair treatment that is performed using the invention may be more or less pronounced, as a function of the initial state of the hair and/or of the desired result. This treatment may especially have the effect of including a removal of heterogeneities present at the surface of the hair, especially via an action that may be termed abrasive, and, as a result, homogenizing the outer surface of the hair.

This type of abrasion may be relatively mild and/or short-lasting, so as to avoid damaging the hair, for example during subsequent treatment or mechanical stresses such as styling.

By virtue of the invention, the hair may be visibly smoother and the effect of the treatment is long-lasting.

Without wishing to be bound by any theory, it may be thought that, by virtue of the invention, the hair may be freed of any deposits present on its surface before the abrasion, and the edges of the scales of the cuticle may be made more regular, which allows increased efficacy of the cationic surfactants comprising at least one ester function.

Furthermore, after treatment, products intended for reinforcing certain properties of the hair or for modifying its appearance can penetrate more easily and deeply into the hair thus treated.

Haircare Composition

Pumice Particles

Pumice is of volcanic origin. It is formed at temperatures from about 500 to 600° C. from lava projected into the air, which cools on falling, and whose degassing leads to the formation of bubbles, resulting in a low density and high porosity.

Pumice is formed from fragments of rhyolite, dacite or andesite. It is considered as a glass since it does not have a crystalline structure.

Pumice particles are abrasive solid particles. In particular, they may have a hardness of greater than or equal to that of the hair, ranging from 3 to 10 Moh, or even greater than or equal to 4, for example greater than or equal to 5, in particular ranging from 5 to 5.5 on the Moh scale.

The pumice particles may have a mean volume diameter of less than or equal to 500 μm, preferably between 50 and 500 μm and better still less than or equal to 300 μm, for example between 100 and 300 μm.

According to the range of particles used, the mean volume diameter may be determined by using screens or by laser granulometry.

It may especially be a pumice powder sold under the name Ponce 0½ D by the company Eyraud, with a mean diameter D [4.3] (mean volume diameter) of about 140 μm measured by laser scattering.

It may also be a decontaminated pumice powder sold under the reference 0-D Ponce by the company Eyraud, with a mean volume diameter of less than 125 μm, or alternatively a pumice powder sold under the reference 2B D by the company Eyraud, with a mean volume diameter ranging from 100 to 500 μm.

The haircare composition in accordance with the invention may comprise pumice particles in a content ranging from 0.1% to 35% by weight, especially from 5% to 30% by weight, in particular from 10% to 25% by weight, for example from 15% to 20% by weight and better still from 18% to 20% by weight, relative to the total weight of the composition.

Cationic Surfactants Comprising at Least One Ester Function

As examples of cationic surfactants comprising at least one ester function, mention may be made especially of quaternary ammonium salts containing at least one ester function of formula (I) below:

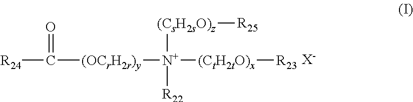

in which:

R₂₂ is chosen from C₁-C₆ alkyl radicals and C₁-C₆ hydroxyalkyl or dihydroxyalkyl radicals;

R₂₃ is chosen from
the radical

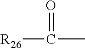

linear or branched, saturated or unsaturated C₁-C₂₂ hydrocarbon-based radicals R₂₇, and
a hydrogen atom,

R₂₅ is selected from:
the radical

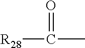

linear or branched, saturated or unsaturated C₁-C₆ hydrocarbon-based radicals R₂₉, and
a hydrogen atom,

R₂₄, R₂₆ and R₂₈, which may be identical or different, are selected from linear or branched, saturated or unsaturated C₇-C₂₁ hydrocarbon-based radicals;

r, s and t, which may be identical or different, are integers ranging from 2 to 6;

y is an integer ranging from 1 to 10;

x and z, which may be identical or different, are integers ranging from 0 to 10; and

X⁻ is a simple or complex, organic or inorganic anion;

with the proviso that the sum x+y+z is from 1 to 15, that when x is 0 then R₂₃ denotes R₂₇, and that when z is 0 then R₂₅ denotes R₂₉.

The alkyl radicals R₂₂ may be linear or branched, and more particularly linear.

Preferably, R₂₂ denotes a methyl, ethyl, hydroxyethyl or dihydroxypropyl radical and more particularly a methyl or ethyl radical.

Advantageously, the sum x+y+z is from 1 to 10.

When R₂₃ is a hydrocarbon radical R₂₇, it may be long and have from 12 to 22 carbon atoms, or may be short and have from 1 to 3 carbon atoms.

When R₂₅ is a hydrocarbon radical R₂₉, it has preferably 1 to 3 carbon atoms.

Advantageously, R₂₄, R₂₆ and R₂₈, which are identical or different, are selected from saturated or unsaturated, linear or branched C₁₁-C₂₁ hydrocarbon radicals, and more particularly from saturated or unsaturated, linear or branched, C₁₁-C₂₁ alkyl and alkenyl radicals.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion is preferably a halide (chloride, bromide or iodide) or an alkyl sulfate, more particularly methyl sulfate. However, methanesulfonate, phosphate, nitrate, tosylate, an anion derived from an organic acid, such as acetate or lactate, or any other anion that is compatible with the ammonium containing an ester function, may be used.

The anion X⁻ is even more particularly chloride or methyl sulfate.

Use is made more particularly in the composition according to the invention of the ammonium salts of formula (I) in which:
R₂₂ denotes a methyl or ethyl radical,
x and y are equal to 1;
z is equal to 0 or 1;
r, s and t are equal to 2;
R₂₃ is chosen from:
   the radical

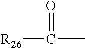

methyl, ethyl or C₁₄-C₂₂ hydrocarbon-based radicals, and
a hydrogen atom;
R₂₅ is selected from:
   the radical

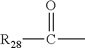

and
   a hydrogen atom;
   R₂₄, R₂₆ and R₂₈, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Mention may be made, for example, of the compounds of formula (I) such as diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium salts (chloride or methyl sulfate, in particular) and mixtures thereof.

The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, for instance the product Dehyquart F30, Stepanquat® by the company Stepan, Noxamium® by the company Ceca, or Rewoquat® WE 18 by the company Rewo-Witco.

The composition according to the invention preferably contains a mixture of quaternary ammonium salts of mono-, di- and triesters with a weight majority of diester salts.

Examples of mixtures of ammonium salts that may be used include the mixture containing from 15% to 30% by weight of acyloxyethyldihydroxyethylmethylammonium methyl sulfate, from 45% to 60% of diacyloxyethylhydroxyethylmethylammonium methyl sulfate and from 15% to 30% of triacyloxyethylmethylammonium methyl sulfate, the acyl radicals containing from 14 to 18 carbon atoms and being derived from palm oil that is optionally partially hydrogenated.

Use may also be made of the ammonium salts containing at least one ester function that are described in U.S. Pat. Nos. 4,874,554 and 4,137,180.

Preferably, the cationic surfactant comprising at least one ester function is a diacyloxydialkylhydroxyalkylammonium salt or a diacyloxytrialkylammonium salt and in particular dipalmitoylethylhydroxyethylammonium methosulfate or dicetearoylethylhydroxyethylmethylammonium methosulfate or distearoylethyldimethylamnionium chloride or distearoyldiethylmethylammonium chloride, and preferably dipalmitoylethylhydroxyethylammonium methosulfate.

The cationic surfactant(s) comprising at least one ester function used in the composition according to the present invention may be present in the composition in a content ranging from 0.1% to 15% by weight, preferably from 0.1% to 10% by weight, and better still from 0.1% to 5% by weight, relative to the total weight of the composition.

The weight ratio of the amount of pumice particles to the amount of cationic surfactants comprising at least one ester function preferably ranges from 1 to 250, even more preferentially from 5 to 100 and better still from 10 to 50.

Nonsilicone Polymer

The haircare composition of the invention also comprises one or more nonsilicone polymers.

For the purposes of the present invention, the term “nonsilicone polymer” means any polymer not comprising any silicon atoms in its structure, and which comprises in said structure a repetition of at least one unit other than an alkylene oxide or glycerol unit.

The nonsilicone polymer(s) may be chosen from nonionic, anionic, cationic or amphoteric thickening, fixing or conditioning polymers.

For the purposes of the present invention, the term “thickening polymer” means any polymer that is capable, by virtue of its presence, of increasing the viscosity of the composition by at least 20 centipoises at 25° C. and at a shear rate of 1 s⁻¹. Even more preferentially, the term “thickening polymer” means a polymer which, when introduced at 1% by weight in an aqueous solution or an aqueous-alcoholic solution containing 30% ethanol, and at pH 7, or in an oil chosen from liquid petroleum jelly, isopropyl myristate or cyclopentadimethylsiloxane, makes it possible to achieve a viscosity of at least 100 cps and preferably of at least 500 cps, at 25° C. and at a shear rate of 1 s⁻¹. This viscosity may be measured using a cone/plate viscometer (Haake R600 rheometer or the like).

For the purposes of the present invention, the term “fixing polymer” means any polymer that makes it possible to give a shape to a head of hair or to hold the hair in a given shape.

For the purposes of the present invention, the term “conditioning polymer” means any polymer which, by virtue of its presence, can improve the cosmetic state of keratin fibers, in particular as regards the feel or the disentangling properties.

The polymers of the invention may be nonionic, anionic, cationic or amphoteric.

More preferentially, the composition of the invention comprises one or more thickening polymers. Even more preferentially, the thickening polymers of the invention are nonionic, anionic, cationic or amphoteric aqueous-phase thickening polymers.

The thickening polymers may be associative or nonassociative polymers.

Thickening polymers according to the invention that may be mentioned include thickening polymers bearing sugar units.

For the purposes of the present invention, the term “sugar unit” means a unit derived from a carbohydrate of formula Cₙ(H₂O)ₙ₋₁ or (CH₂O)ₙ, which may be optionally modified by substitution and/or by oxidation and/or by dehydration.

The sugar units that may be included in the composition of the thickening polymers of the invention are preferably derived from the following sugars: glucose, galactose, arabinose, rhamnose, mannose, xylose, fucose, anhydrogalactose, galacturonic acid, glucuronic acid, mannuronic acid, galactose sulfate, anhydrogalactose sulfate.

Thickening polymers of the invention that may especially be mentioned include:
native gums such as:
   a) tree or shrub exudates, including:
      gum arabic (branched polymer of galactose, arabinose, rhamnose and glucuronic acid);
      ghatti gum (polymer derived from arabinose, galactose, mannose, xylose and glucuronic acid);
      karaya gum (polymer derived from galacturonic acid, galactose, rhamnose and glucuronic acid); and
      gum tragacanth (or tragacanth) (polymer of galacturonic acid, galactose, fucose, xylose and arabinose);
   b) gums derived from algae, including:
      agar (polymer derived from galactose and anhydrogalactose);
      alginates (polymers of mannuronic acid and of glucuronic acid); and
      carrageenans and furcellerans (polymers of galactose sulfate and of anhydrogalactose sulfate);
   c) gums derived from seeds or tubers, including:
      guar gum (polymer of mannose and galactose);
      locust bean gum (polymer of mannose and galactose);
      fenugreek gum (polymer of mannose and galactose);
      tamarind gum (polymer of galactose, xylose and glucose);
      konjac gum (polymer of glucose and mannose);
   d) microbial gums, including:
      xanthan gum (polymer of glucose, mannose acetate, mannose/pyruvic acid and glucuronic acid);
      gellan gum (polymer of partially acylated glucose, rhamnose and glucuronic acid); and
      scleroglucan gum (glucose polymer);
   e) plant extracts, including:
      cellulose (glucose polymer); and
      starch (glucose polymer).

These polymers may be physically or chemically modified. A physical treatment that may especially be mentioned is the temperature.

Chemical treatments that may be mentioned include esterification, etherification, amidation or oxidation reactions. These treatments can lead to polymers that may especially be nonionic, anionic or amphoteric.

Preferably, these chemical or physical treatments are applied to guar gums, locust bean gums, starches and celluloses.

The nonionic guar gums that may be used according to the invention may be modified with C₁-C₆ hydroxyalkyl groups.

Among the hydroxyalkyl groups that may be mentioned, for example, are hydroxymethyl, hydroxyethyl, hydroxypropyl and hydroxybutyl groups.

These guar gums are well known in the prior art and can be prepared, for example, by reacting the corresponding alkene oxides such as, for example, propylene oxides, with the guar gum so as to obtain a guar gum modified with hydroxypropyl groups.

The degree of hydroxyalkylation preferably ranges from 0.4 to 1.2, and corresponds to the number of alkylene oxide molecules consumed by the number of free hydroxyl functions present on the guar gum.

Such nonionic guar gums optionally modified with hydroxyalkyl groups are sold, for example, under the trade names Jaguar HP8, Jaguar HP60 and Jaguar HP120 by the company Rhodia Chimie.

The botanical origin of the starch molecules used in the present invention may be cereals or tubers. Thus, the starches are chosen, for example, from corn starch, rice starch, cassava starch, barley starch, potato starch, wheat starch, sorghum starch and pea starch.

The starches may be chemically or physically modified especially by one or more of the following reactions: pregelatinization, oxidation, crosslinking, esterification, etherification, amidation, heat treatments.

More particularly, these reactions may be performed in the following manner:
pregelatinization by splitting the starch granules (for example drying and cooking in a drying drum);
oxidation with strong oxidizing agents, leading to the introduction of carboxyl groups into the starch molecule and to depolymerization of the starch molecule (for example by treating an aqueous starch solution with sodium hypochlorite);
crosslinking with functional agents capable of reacting with the hydroxyl groups of the starch molecules, which will thus bond together (for example with glyceryl and/or phosphate groups);
esterification in alkaline medium for the grafting of functional groups, especially C₁-C₆ acyl (acetyl), C₁-C₆ hydroxyalkyl (hydroxyethyl or hydroxypropyl), carboxymethyl or octenylsuccinic.

Monostarch phosphates (of the type Am—O—PO—(OX)₂), distarch phosphates (of the type Am—O—PO—(OX)—O—Am) or even tristarch phosphates (of the type Am—O—PO—(O—Am)₂) or mixtures thereof (Am meaning starch) may especially be obtained by crosslinking with phosphorus compounds.

X especially denotes alkali metals (for example sodium or potassium), alkaline-earth metals (for example calcium or magnesium), ammonium salts, amine salts, for instance those of monoethanolamine, diethanolamine, triethanolamine, 3-amino-1,2-propanediol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Distarch phosphates or compounds rich in distarch phosphate will preferentially be used, for instance the products sold under the references Prejel VA-70-T AGGL (gelatinized hydroxypropyl cassava distarch phosphate), Prejel TK1 (gelatinized cassava distarch phosphate) and Prejel 200 (gelatinized acetyl cassava distarch phosphate) by the company Avebe, or Structure Zea from National Starch (gelatinized corn distarch phosphate).

A preferred starch is a starch that has undergone at least one chemical modification such as at least one esterification.

According to the invention, amphoteric starches may also be used, these amphoteric starches comprising one or more anionic groups and one or more cationic groups. The anionic and cationic groups may be linked to the same reactive site of the starch molecule or to different reactive sites; they are preferably linked to the same reactive site. The anionic groups may be of carboxylic, phosphate or sulfate type, preferably carboxylic. The cationic groups may be of primary, secondary, tertiary or quaternary amine type.

The amphoteric starches are especially chosen from the compounds having the following formulae:

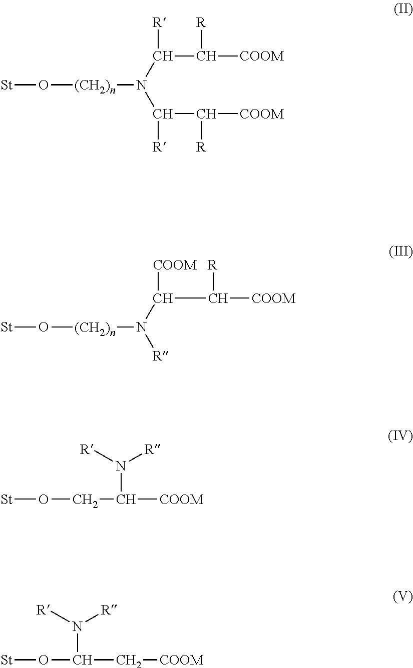

in which formulae:

St-O represents a starch molecule,

R, which may be identical or different, represents a hydrogen atom or a methyl radical,

R′, which may be identical or different, represents a hydrogen atom, a methyl radical or a —COOH group,

n is an integer equal to 2 or 3,

M, which may be identical or different, denotes a hydrogen atom, an alkali metal or alkaline-earth metal such as Na, K, Li or NH₄, a quaternary ammonium or an organic amine,

R″ represents a hydrogen atom or an alkyl radical containing from 1 to 18 carbon atoms.

These compounds are especially described in U.S. Pat. No. 5,455,340 and U.S. Pat. No. 4,017,460, which are included by way of reference.

The starch molecules may be derived from any plant source of starch, especially such as corn, potato, oat, rice, tapioca, sorghum, barley or wheat. It is also possible to use the starch hydrolysates mentioned above. The starch is preferably derived from potato.

The starches of formula (III) or (IV) are particularly used. Starches modified with 2-chloroethylaminodipropionic acid are more particularly used, i.e. starches of formula (III) or (IV) in which R, R′, R″ and M represent a hydrogen atom and n is equal to 2. The preferred amphoteric starch is a starch chloroethylamidodipropionate.

As mentioned previously, the cellulose derivatives may especially be anionic, amphoteric or nonionic.

Among these derivatives, cellulose ethers, cellulose esters and cellulose ester ethers are distinguished.

Among the cellulose esters are inorganic esters of cellulose, for example cellulose nitrates, sulfates, phosphates, organic cellulose esters, for example cellulose monoacetates, triacetates, amidopropionates, acetatebutyrates, acetatepropionates and acetatetrimellitates, and mixed organic/inorganic esters of cellulose, such as cellulose acetatebutyrate sulfates and cellulose acetatepropionate sulfates. Among the cellulose ester ethers, mention may be made of hydroxypropylmethylcellulose phthalates and ethylcellulose sulfates.

Among the nonionic cellulose ethers that may be mentioned are alkylcelluloses such as methylcelluloses and ethylcelluloses (for example Ethocel Standard 100 Premium from Dow Chemical); hydroxyalkylcelluloses such as hydroxymethylcelluloses and hydroxyethylcelluloses (for example Natrosol 250 HHR sold by Aqualon) and hydroxypropylcelluloses (for example Klucel EF from Aqualon); mixed hydroxyalkyl-alkylcelluloses such as hydroxypropylmethylcelluloses (for example Methocel E4M from Dow Chemical), hydroxyethylmethylcelluloses, hydroxyethylethylcelluloses (for example Bermocoll E 481 FQ from Akzo Nobel) and hydroxybutylmethylcelluloses.

Among the anionic cellulose ethers, mention may be made of carboxyalkylcelluloses and salts thereof. Examples that may be mentioned include carboxymethylcelluloses, carboxymethylmethylcelluloses (for example Blanose 7M from the company Aqualon) and carboxymethylhydroxyethylcelluloses, and the sodium salts thereof.

Among the nonassociative thickening polymers not bearing sugar units that may be used, mention may be made of crosslinked acrylic or methacrylic acid homopolymers or copolymers, crosslinked 2-acrylamido-2-methylpropanesulfonic acid homopolymers and crosslinked acrylamide copolymers thereof, ammonium acrylate homopolymers, or copolymers of ammonium acrylate and of acrylamide, alone or mixtures thereof.

A first family of nonassociative thickening polymers that is suitable for use is represented by crosslinked acrylic acid homopolymers.

Among the homopolymers of this type, mention may be made of those crosslinked with an allyl alcohol ether of the sugar series, such as, for example, the products sold under the names Carbopol 980, 981, 954, 2984 and 5984 by the company Noveon or the products sold under the names Synthalen M and Synthalen K by the company 3 VSA.

The nonassociative thickening polymers may also be crosslinked (meth)acrylic acid copolymers, such as the polymer sold under the name Aqua SF1 by the company Noveon.

The nonassociative thickening polymers may be chosen from crosslinked 2-acrylamido-2-methylpropanesulfonic acid homopolymers and the crosslinked acrylamide copolymers thereof.

As regards these homopolymers and copolymers, which may be partially or totally neutralized, mention may be made of polymers comprising from 90% to 99.9% by weight, relative to the total weight of the polymer, of units of formula (j) below:

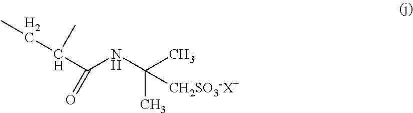

in which X⁺ denotes a cation or a mixture of cations, or a proton.

More particularly, the cations are chosen from alkali metals (for instance sodium or potassium), ammonium ions optionally substituted with 1 to 3 alkyl radicals, which may be identical or different, containing from 1 to 6 carbon atoms, optionally bearing at least one hydroxyl radical, cations derived from N-methylglucamine or from basic amino acids, for instance arginine and lysine. Preferably, the cation is an ammonium or sodium ion.

Moreover, the polymer comprises from 0.01% to 10% by weight, relative to the total weight of the polymer, of crosslinking units derived from at least one monomer containing at least two ethylenic unsaturations (carbon-carbon double bond).

The crosslinking monomers containing at least two ethylenic unsaturations are chosen, for example, from diallyl ether, triallyl cyanurate, diallyl maleate, allyl (meth)acrylate, dipropylene glycol diallyl ether, polyglycol diallyl ethers, triethylene glycol divinyl ether, hydroquinone diallyl ether, tetraallyloxyethane, tetra- or diethylene glycol di(meth)acrylate, triallylamine, tetraallylethylenediamine, trimethyloipropane diallyl ether, trimethylolpropane triacrylate, methylenebis(meth)acrylamide or divinylbenzene, allylic ethers of alcohols of the sugar series, or other allylic or vinyl ethers of polyfunctional alcohols, and also allylic esters of phosphoric and/or vinylphosphonic acid derivatives, or mixtures of these compounds.

For further details regarding these polymers, reference may be made to document EP 0 815 828.

Among the partially or totally neutralized crosslinked copolymers of 2-acrylamido-2-methylpropanesulfonic acid and of acrylamide, mention may be made in particular of the product described in Example 1 of document EP 0 503 853, and reference may be made to said document as regards these polymers.

The composition may similarly comprise, as nonassociative thickening polymers, ammonium acrylate homopolymers or copolymers of ammonium acrylate and of acrylamide.

Among the ammonium acrylate homopolymers that may be mentioned is the product sold under the name Microsap PAS 5193 by the company Hoechst. Among the copolymers of ammonium acrylate and of acrylamide that may be mentioned is the product sold under the name Bozepol C Nouveau or the product PAS 5193 sold by the company Hoechst. Reference may be made especially to documents FR 2 416 723, U.S. Pat. No. 2,798,053 and U.S. Pat. No. 2,923,692 as regards the description and preparation of such compounds.

Among the thickeners, mention may also be made of thickening systems based on associative polymers that are well known to those skilled in the art and especially of nonionic, anionic, cationic or amphoteric nature.

It is recalled that associative polymers are polymers that are capable, in an aqueous medium, of reversibly associating with each other or with other molecules.

Their chemical structure more particularly comprises at least one hydrophilic region and at least one hydrophobic region.

The term “hydrophobic group” means a radical or polymer with a saturated or unsaturated, linear or branched hydrocarbon-based chain, comprising at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a monofunctional compound. By way of example, the hydrophobic group may be derived from a fatty alcohol such as stearyl alcohol, dodecyl alcohol or decyl alcohol. It may also denote a hydrocarbon-based polymer, for instance polybutadiene.

Among the associative polymers of anionic type that may be mentioned are:
(I) those comprising at least one hydrophilic unit and at least one fatty-chain allyl ether unit, more particularly those whose hydrophilic unit is formed by an ethylenic unsaturated anionic monomer, more particularly a vinylcarboxylic acid and most particularly an acrylic acid or a methacrylic acid or mixtures thereof, the fatty-chain allyl ether unit of which corresponds to the monomer of formula (VI) below:

CH₂═CR′CH₂OBₙR (VI)

in which R′ denotes H or CH₃, B denotes an ethylenoxy radical, n is zero or denotes an integer ranging from 1 to 100, R denotes a hydrocarbon-based radical chosen from alkyl, arylalkyl, aryl, alkylaryl and cycloalkyl radicals, containing from 8 to 30 carbon atoms, preferably 10 to 24 carbon atoms and even more particularly from 12 to 18 carbon atoms. A unit of formula (VI) that is more particularly preferred is a unit in which R′ denotes H, n is equal to 10 and R denotes a stearyl (C₁₈) radical.

Anionic associative polymers of this type are described and prepared, according to an emulsion polymerization process, in patent EP-0 216 479.

Among these anionic associative polymers that are particularly preferred according to the invention are polymers formed from 20% to 60% by weight of acrylic acid and/or of methacrylic acid, from 5% to 60% by weight of lower alkyl (meth)acrylates, from 2% to 50% by weight of fatty-chain allyl ether of formula (VI), and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, those most particularly preferred are crosslinked terpolymers of methacrylic acid, of ethyl acrylate and of polyethylene glycol (10 EO) stearyl alcohol ether (Steareth-10), in particular those sold by the company Ciba under the names Salcare SC 80® and Salcare SC 90®, which are aqueous 30% emulsions of a crosslinked terpolymer of methacrylic acid, of ethyl acrylate and of steareth-10 allyl ether (40/50/10);
(II) those comprising at least one hydrophilic unit of unsaturated olefinic carboxylic acid type, and at least one hydrophobic unit of (C₁₀-C₃₀)alkyl ester of unsaturated carboxylic acid type.

These polymers are preferably chosen from those in which the hydrophilic unit of unsaturated olefinic carboxylic acid type corresponds to the monomer of formula (VII) below:

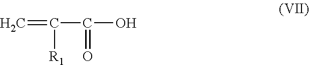

in which. R₁ denotes H, CH₃, or C₂H₅, i.e. acrylic acid, methacrylic acid or ethacrylic acid units, and in which the hydrophobic unit of (C₁₀-C₃₀)alkyl ester of unsaturated carboxylic acid type corresponds to the monomer of formula (VIII) below:

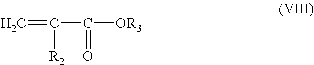

in which R₂ denotes H or CH₃ or C₂H₅ (i.e. acrylate, methacrylate or ethacrylate units) and preferably H (acrylate units) or CH₃ (methacrylate units), R₃ denoting a C₁₀-C₃₀ and preferably C₁₂C₂₂ alkyl radical.

(C₁₀-C₃₀) alkyl esters of unsaturated carboxylic acids according to the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic polymers of this type are described and prepared, for example, according to U.S. Pat. No. 3,915,921 and U.S. Pat. No. 4,509,949.

Among the anionic associative polymers of this type that will be used more particularly are polymers formed from a monomer mixture comprising:

(i) essentially acrylic acid,

(ii) an ester of formula (VIII) described above and in which R₂ denotes H or CH₃, R₃ denoting an alkyl radical containing from 12 to 22 carbon atoms, and

(iii) a crosslinking agent, which is a well-known copolymerizable unsaturated polyethylenic monomer, for instance diallyl phthalate, allyl (meth)acrylate, divinylbenzene, (poly)ethylene glycol dimethacrylate and methylenebisacrylamide.

Among anionic associative polymers of this type that will be used more particularly are those consisting of from 60% to 95% by weight of acrylic acid (hydrophilic unit), 4% to 40% by weight of C₁₀-C₃₀ alkyl acrylate (hydrophobic unit) and 0 to 6% by weight of crosslinking polymerizable monomer, or alternatively those consisting of from 96% to 98% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer such as those described above.

Among said above polymers, those most particularly preferred according to the present invention are the products sold by the company Goodrich under the trade names Pemulen TR1®, Pemulen TR2® and Carbopol 1382®, and even more preferentially Pemulen TR1®, and the product sold by the company SEPPIC under the name Coatex SX®.

Mention may also be made of polymers which, besides the monomers of formula (VII) and of formula (VIII), contain one or more other monomers. This additional monomer may especially be a vinyllactam and in particular vinylpyrrolidone.

An example of a polymer that may be mentioned is the acrylic acid/lauryl methacrylate/vinylpyrrolidone terpolymer sold under the name Acrylidone LM by the company ISP;
(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.
(IV) acrylic terpolymers comprising:

(a) about 20% to 70% by weight of an α,β-monoethylenically unsaturated carboxylic acid,

(b) about 20% to 80% by weight of an α,β-monoethylenically unsaturated non-surfactant monomer other than (a), and

(c) about 0.5% to 60% by weight of a nonionic monourethane which is the product of reaction of a monohydric surfactant with a monoethylenically unsaturated monoisocyanate,

such as those described in patent application EP-A-0 173 109 and more particularly the terpolymer described in Example 3, namely a methacrylic acid/methyl acrylate/behenyl alcohol dimethyl-meta-isopropenylbenzylisocyanate ethoxylated (40 EO) terpolymer, as an aqueous 25% dispersion.
(V) copolymers comprising among their monomers an α,β-monoethylenically unsaturated carboxylic acid and an ester of an α,β-monoethylenically unsaturated carboxylic acid and of an oxyalkylenated fatty alcohol.

Preferentially, these compounds also comprise as monomer an ester of an α,β-monoethylenically unsaturated carboxylic acid and of a C₁-C₄ alcohol.

An example of a compound of this type that may be mentioned is Aculyn 22® sold by the company Röhm & Haas, which is a methacrylic acid/ethyl acrylate/oxyalkylenated stearyl methacrylate terpolymer; and
(VI) amphiphilic polymers comprising at least one ethylenically unsaturated monomer bearing a sulfonic group, in free or partially or totally neutralized form and comprising at least one hydrophobic part. These polymers may be crosslinked or noncrosslinked. They are preferably crosslinked.

The ethylenically unsaturated monomers bearing a sulfonic group are especially chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids such as undecylacrylamidomethanesulfonic acid, and also partially or totally neutralized forms thereof, and mixtures thereof.

(Meth)acrylamido(C₁-C₂₂)alkylsulfonic acids, for instance acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof, will more preferentially be used.

2-Acrylamido-2-methylpropanesulfonic acid (AMPS), and also partially or totally neutralized forms thereof, will more particularly be used.

The polymers of this family may be chosen especially from random amphiphilic AMPS polymers modified by reaction with a C₆-C₂₂ n-monoalkylamine or di-n-alkylamine, and such as those described in patent application WO 00/31154, which form an integral part of the content of the description. These polymers may also contain other ethylenically unsaturated hydrophilic monomers chosen, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The preferred polymers of this family are chosen from amphiphilic copolymers of AMPS and of at least one ethylenically unsaturated hydrophobic monomer.

These same copolymers may also contain one or more ethylenically unsaturated monomers not comprising a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described especially in patent application EP-A-0 750 899, U.S. Pat. No. 5,089,578 and in the following publications from Yotaro Morishima:
“Self-assembling amphiphilic polyelectrolytes and their nanostructures—Chinese Journal of Polymer Science Vol. 18, No. 40, (2000), 323-336”;
“Micelle formation of random copolymers of sodium 2-(acrylamido)-2-methylpropanesulfonate and a nonionic surfactant macromonomer in water as studied by fluorescence and dynamic light scattering—Macromolecules 2000, Vol. 33, No. 10-3694-3704”;
“Solution properties of micelle networks formed by nonionic moieties covalently bound to a polyelectrolyte: salt effects on rheological behavior—Langmuir, 2000, Vol. 16, No. 12, 5324-5332”;
“Stimuli responsive amphiphilic copolymers of sodium 2-(acrylamido)-2-methylpropanesulfonate and associative macromonomers—Polym. Preprint, Div. Polym. Chem. 1999, 40(2), 220-221”.

The ethylenically unsaturated hydrophobic monomers of these particular copolymers are preferably chosen from the acrylates or acrylamides of formula (IX) below:

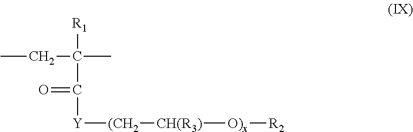

in which R₁ and R₃, which may be identical or different, denote a hydrogen atom or a linear or branched C₁-C₆ alkyl radical (preferably methyl); Y denotes O or NH; R₂ denotes a hydrophobic hydrocarbon-based radical containing at least 8 and more preferentially from 8 to 22 carbon atoms, even more preferentially from 8 to 18 carbon atoms and more particularly from 12 to 18 carbon atoms; x denotes a number of moles of alkylene oxide and ranges from 0 to 100.

The radical R₂ is preferably chosen from saturated or unsaturated linear C₈-C₁₈ alkyl radicals (for example n-hexyl, n-octyl, n-decyl, n-hexadecyl, n-dodecyl), branched or cyclic (for example cyclododecane (C₁₂) or adamantane (C₁₀) alkyl radicals); C₆-C₁₈ perfluoroalkyl radicals (for example the group of formula —(CH₂)₂—(CF₂)₉—CF₃); the cholesteryl radical (C₂₇) or a cholesterol ester, for instance the cholesteryl oxyhexanoate group; polycyclic aromatic groups, for instance naphthalene or pyrene. Among these radicals, the ones that are more particularly preferred are linear alkyl radicals and more particularly the n-dodecyl radical.

According to one particularly preferred form of the invention, the monomer of formula (IX) comprises at least one alkylene oxide unit (x≧1) and preferably a polyoxyalkylene chain. The polyoxyalkylene chain is preferentially formed from ethylene oxide units and/or propylene oxide units and even more particularly formed from ethylene oxide units. The number of oxyalkylene units generally ranges from 3 to 100, more preferentially from 3 to 50, even more preferentially from 7 to 25 and better still from 8 to 25.

Among these polymers, mention may be made of:
crosslinked or noncrosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer, such as those described in patent application EP-A 0 750 899;
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of copolymers of totally neutralized AMPS and of dodecyl methacrylate, and also crosslinked and noncrosslinked copolymers of AMPS and of n-dodecylmethacrylamide, such as those described in the Morishima articles mentioned above.

Mention will be made more particularly of the copolymers formed from 2-acrylamido-2-methylpropanesulfonic acid (AMPS) units of formula (X) below:

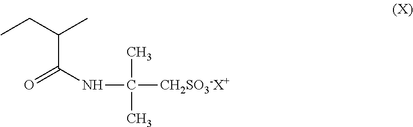

in which X⁺ is a proton, an alkali metal cation, an alkaline-earth metal cation or the ammonium ion,

and units of formula (XI) below:

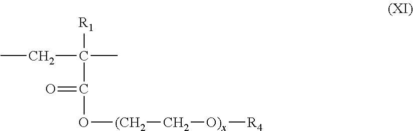

in which x denotes an integer ranging from 3 to 100, preferably from 5 to 80 and more preferentially from 7 to 25; R₁ has the same meaning as that indicated above in formula (IX) and R₄ denotes a linear or branched C₆-C₂₂, more preferentially C₁₀-C₂₂ and better still C₁₂-C₁₈ alkyl.

The polymers that are particularly preferred are those for which x=25, R₁ denotes methyl and R₄ represents n-dodecyl; they are described in the Morishima articles mentioned above.

The molar percentage concentration of units of formula (X) and of units of formula (XI) will vary as a function of the desired cosmetic application and of the rheological properties sought for the formulation. It will preferably range from 70 mol % to 99 mol % of AMPS units and from 1 mol % to 30 mol % of units of formula (XI) relative to the copolymer, and more particularly from 70 mol % to 90 mol % of AMPS units and from 10 mol % to 30 mol % of units of formula (XI).

The polymers for which X⁺ denotes the sodium ion or the ammonium ion are more particularly preferred.

Among the associative polymers of cationic type that may be mentioned are:
(I) cationic associative polyurethanes, the family of which has been described by the Applicant in French patent application No. 00/09609; it may be represented by the general formula (XII) below:

R—X—(P)ₙ-[L-(Y)ₘ]ᵣ-L′-(P′)ₚ—X′—R′ (XII)

in which:

R and R′, which may be identical or different, represent a hydrophobic group or a hydrogen atom;

X and X′, which may be identical or different, represent a group comprising an amine function optionally bearing a hydrophobic group, or alternatively a group L″;

L, L′ and L″, which may be identical or different, represent a group derived from a diisocyanate;

P and P′, which may be identical or different, represent a group comprising an amine function optionally bearing a hydrophobic group;

Y represents a hydrophilic group;

r is an integer between 1 and 100, preferably between 1 and 50 and in particular between 1 and 25;

n, m and p each range, independently of each other, from 0 to 1000;

the molecule containing at least one protonated or quaternized amine function and at least one hydrophobic group.

In one preferred embodiment of these polyurethanes, the only hydrophobic groups are the groups R and R′ at the chain ends.

One preferred family of cationic associative polyurethanes is the one corresponding to formula (XII) described above and in which:

R and R′ both independently represent a hydrophobic group,
X and X′ each represent a group L″,
n and p are between 1 and 1000, and
L, L′, L″, P, P′, Y and m have the meaning given above.

Another preferred family of cationic associative polyurethanes is the one corresponding to formula (XII) above in which:

R and R′ both independently represent a hydrophobic group, X and X′ each represent a group L″, n and p are 0, and L, L′, L″, Y and m have the meaning given above.

The fact that n and p are 0 means that these polymers do not comprise units derived from a monomer containing an amine function, incorporated into the polymer during the polycondensation. The protonated amine functions of these polyurethanes result from the hydrolysis of excess isocyanate functions, at the chain end, followed by alkylation of the primary amine functions formed with alkylating agents containing a hydrophobic group, i.e. compounds of the type RQ or R′Q, in which R and R′ are as defined above and Q denotes a leaving group such as a halide, a sulfate, etc.

Yet another preferred family of cationic associative polyurethanes is the one corresponding to formula (XII) above in which:

R and R′ both independently represent a hydrophobic group,

X and X′ both independently represent a group comprising a quaternary amine,

n and p are zero, and

L, L′, Y and m have the meaning given above.

The number-average molecular mass of the cationic associative polyurethanes is preferably between 400 and 500000, in particular between 1000 and 400000 and ideally between 1000 and 300000.

The expression “hydrophobic group” means a radical or polymer containing a saturated or unsaturated, linear or branched hydrocarbon-based chain, which may contain one or more heteroatoms such as P, O, N or S, or a radical containing a perfluoro or silicone chain. When the hydrophobic group denotes a hydrocarbon-based radical, it comprises at least 10 carbon atoms, preferably from 10 to 30 carbon atoms, in particular from 12 to 30 carbon atoms and more preferentially from 18 to 30 carbon atoms.

Preferentially, the hydrocarbon-based group is derived from a mono functional compound.

By way of example, the hydrophobic group may be derived from a fatty alcohol such as stearyl alcohol, dodecyl alcohol or decyl alcohol. It may also denote a hydrocarbon-based polymer, for instance polybutadiene.

When X and/or X′ denote(s) a group comprising a tertiary or quaternary amine, X and/or X′ may represent one of the following formulae:

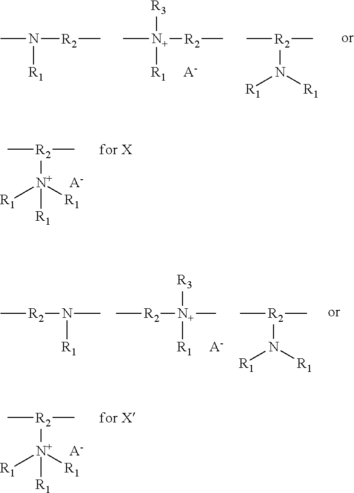

in which:

R₂ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P;

R₁ and R₃, which may be identical or different, denote a C₁-C₃₀ alkyl or alkenyl radical or an aryl radical, at least one of the carbon atoms possibly being replaced with a heteroatom chosen from N, S, O and P;

A⁻ is a physiologically acceptable counterion.

The groups L, L′ and L″ represent a group of formula:

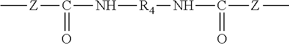

in which:

Z represents —O—, —S— or —NH—; and

R₄ represents a linear or branched alkylene radical containing from 1 to 20 carbon atoms, optionally comprising a saturated or unsaturated ring, or an arylene radical, one or more of the carbon atoms possibly being replaced with a heteroatom chosen from N, S, O and P.

The groups P and P′ comprising an amine function may represent at least one of the following formulae:

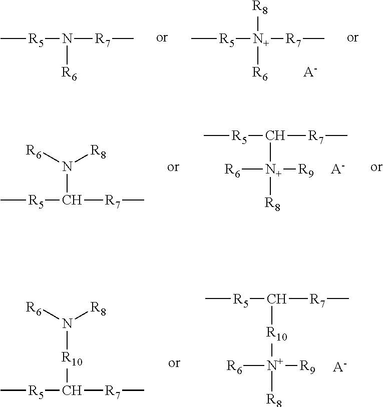

in which:

R₅ and R₇ have the same meanings as R₂ defined above;

R₆, R₈ and R₉ have the same meanings as R₁ and R₃ defined above;

R₁₀ represents a linear or branched, optionally unsaturated alkylene group possibly containing one or more heteroatoms chosen from N, O, S and P;

and A⁻ is a physiologically acceptable counterion.

As regards the meaning of Y, the term “hydrophilic group” means a polymeric or nonpolymeric water-soluble group.

By way of example, when it is not a polymer, mention may be made of ethylene glycol, diethylene glycol and propylene glycol.

When it is a hydrophilic polymer, in accordance with one preferred embodiment, mention may be made, for example, of polyethers, sulfonated polyesters, sulfonated polyamides or a mixture of these polymers. The hydrophilic compound is preferentially a polyether and especially a poly(ethylene oxide) or poly(propylene oxide).

The cationic associative polyurethanes of formula (XII) that may be used according to the invention are formed from diisocyanates and from various compounds bearing functions containing a labile hydrogen. The functions containing a labile hydrogen may be alcohol, primary or secondary amine or thiol functions, giving, after reaction with the diisocyanate functions, polyurethanes, polyureas and polythioureas, respectively. The expression “polyurethanes that may be used according to the present invention” encompasses these three types of polymer, namely polyurethanes per se, polyureas and polythioureas, and also copolymers thereof.

A first type of compound involved in the preparation of the polyurethane of formula (XII) is a compound comprising at least one unit containing an amine function. This compound may be multifunctional, but the compound is preferentially difunctional, that is to say that, according to one preferential embodiment, this compound comprises two labile hydrogen atoms borne, for example, by a hydroxyl, primary amine, secondary amine or thiol function. A mixture of multifunctional and difunctional compounds in which the percentage of multifunctional compounds is low may also be used.

As mentioned above, this compound may comprise more than one unit containing an amine function. In this case, it is a polymer bearing a repetition of the unit containing an amine function.

Compounds of this type may be represented by one of the following formulae:

HZ—(P)ₙ—ZH,

or

HZ—(P′)ₚ—ZH

in which Z, P, P′, n and p are as defined above.

Examples of compounds containing an amine function that may be mentioned include N-methyldiethanolamine, N-tert-butyldiethanolamine and N-sulfoethyldiethanolamine.

The second compound included in the preparation of the polyurethane of formula (XII) is a diisocyanate corresponding to the formula:

O═C═N—R₄—N═C═O

in which R₄ is as defined above.

By way of example, mention may be made of methylenediphenyl diisocyanate, methylenecyclohexane diisocyanate, isophorone diisocyanate, tolylene diisocyanate, naphthalene diisocyanate, butane diisocyanate and hexane diisocyanate.

A third compound involved in the preparation of the polyurethane of formula (XII) is a hydrophobic compound intended to form the terminal hydrophobic groups of the polymer of formula (XII).

This compound is formed from a hydrophobic group and a function containing a labile hydrogen, for example a hydroxyl, primary or secondary amine, or thiol function.

By way of example, this compound may be a fatty alcohol such as, in particular, stearyl alcohol, dodecyl alcohol or decyl alcohol. When this compound comprises a polymeric chain, it may be, for example, α-hydroxylated hydrogenated polybutadiene.

The hydrophobic group of the polyurethane of formula (XII) may also result from the quaternization reaction of the tertiary amine of the compound comprising at least one tertiary amine unit. Thus, the hydrophobic group is introduced via the quaternizing agent. This quaternizing agent is a compound of the type RQ or R′Q, in which R and R′ are as defined above and Q denotes a leaving group such as a halide; a sulfate, etc.

The cationic associative polyurethane may also comprise a hydrophilic block. This block is provided by a fourth type of compound involved in the preparation of the polymer. This compound may be multifunctional. It is preferably difunctional. It is also possible to have a mixture in which the percentage of multifunctional compound is low.

The functions containing a labile hydrogen are alcohol, primary or secondary amine or thiol functions. This compound may be a polymer terminated at the chain ends with one of these functions containing a labile hydrogen.

By way of example, when it is not a polymer, mention may be made of ethylene glycol, diethylene glycol and propylene glycol.

When it is a hydrophilic polymer, mention may be made, for example, of polyethers, sulfonated polyesters and sulfonated polyamides, or a mixture of these polymers. The hydrophilic compound is preferentially a polyether and especially a poly(ethylene oxide) or poly(propylene oxide).

The hydrophilic group termed Y in formula (XII) is optional. Specifically, the units containing a quaternary amine or protonated function may suffice to provide the solubility or water-dispersibility required for this type of polymer in an aqueous solution.

Although the presence of a hydrophilic group Y is optional, cationic associative polyurethanes comprising such a group are, however, preferred.
(II) the cationic polymer(s) obtained by polymerization of a monomer mixture comprising one or more vinyl monomers substituted with one or more amino groups, one or more hydrophobic nonionic vinyl monomers, and one or more associative vinyl monomers.

In particular, among these cationic polymers, mention may be made especially of the compound sold by the company Noveon under the name Aqua CC and which corresponds to the INCI name Polyacrylate-1 Crosspolymer.

Polyacrylate-1 Crosspolymer is the product of polymerization of a monomer mixture comprising:
a di(C₁-C₄ alkyl)amino(C₁-C₆ alkyl)methacrylate,
one or more C₁-C₃₀ alkyl esters of (meth)acrylic acid,
a polyethoxylated C₁₀-C₃₀ alkyl methacrylate (20-25 mol of ethylene oxide units),
a 30/5 polyethylene glycol/polypropylene glycol allyl ether,
a hydroxy(C₂-C₆ alkyl)methacrylate, and
an ethylene glycol dimethacrylate.
quaternized alkylhydroxyethylcelluloses (cationic), such as the products Quatrisoft LM 200, Quatrisoft LM-X 529-18-A, Quatrisoft LM-X 529-18-B (C₁₂ alkyl) and Quatrisoft LM-X 529-8 (C₁₈ alkyl) sold by the company Amerchol, the products Crodacel QM and Crodacel QL (C₁₂ alkyl) and Crodacel QS (C₁₈ alkyl) sold by the company Croda, and the product Softcat SL 100 sold by the company Amerchol.
(IV) cationic polyvinyllactam polymers comprising:
a) at least one monomer of vinyllactam or alkylvinyllactam type;
b) at least one monomer of structure (XIII) or (XIV) below:

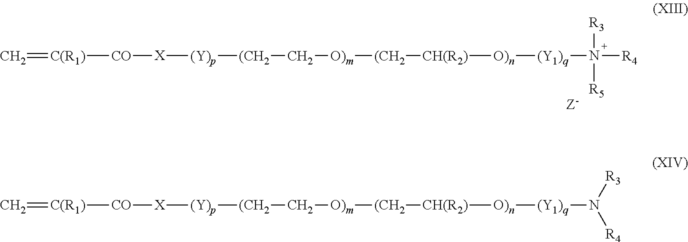

in which:

X denotes an oxygen atom or a radical NR₆,

R₁ and R₆ denote, independently of each other, a hydrogen atom or a linear or branched C₁-C₅ alkyl radical,

R₂ denotes a linear or branched C₁-C₄ alkyl radical,

R₃, R₄ and R₅ denote, independently of each other, a hydrogen atom, a linear or branched C₁-C₃₀ alkyl radical or a radical of formula (XV):

—(Y₂)ᵣ—(CH₂—CH(R₇)—O)ₓ—R₈ (XV)

Y, Y₁ and Y₂ denote, independently of each other, a linear or branched C₂-C₁₆ alkylene radical,

R₇ denotes a hydrogen atom or a linear or branched C₁-C₄ alkyl radical or a linear or branched C₁-C₄ hydroxyalkyl radical,

R₈ denotes a hydrogen atom or a linear or branched C₁-C₃₀ alkyl radical,

p, q and r denote, independently of each other, either the value 0 or the value 1,

m and n denote, independently of each other, an integer ranging from 0 to 100,

x denotes an integer ranging from 1 to 100,

Z denotes an organic or mineral acid anion, with the proviso that:
at least one of the substituents R₃, R₄, R₅ or R₈ denotes a linear or branched C₉-C₃₀ alkyl radical,
if m or n is other than zero, then q is equal to 1,
if m and n are equal to zero, then p or q is equal to 0.

The cationic poly(vinyllactam) polymers according to the invention may be crosslinked or noncrosslinked and may also be block polymers.

Preferably, the counterion Z⁻ of the monomers of formula (XIII) is chosen from halide ions, phosphate ions, the methosulfate ion and the tosylate ion.

Preferably, R₃, R₄ and R₅ denote, independently of each other, a hydrogen atom or a linear or branched C₁-C₃₀ alkyl radical.

More preferentially, the monomer b) is a monomer of formula (XII) for which, even more preferentially, m and n are equal to 0.

The vinyllactam or alkylvinyllactam monomer is preferably a compound of structure (XVI):

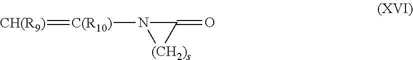

in which:

s denotes an integer ranging from 3 to 6,

R₉ denotes a hydrogen atom or a C₁-C₃ alkyl radical,

R₁₀ denotes a hydrogen atom or a C₁-C₅ alkyl radical,

with the proviso that at least one of the radicals R₉ and R₁₀ denotes a hydrogen atom.

Even more preferentially, the monomer (XVI) is vinylpyrrolidone.

The cationic poly(vinyllactam) polymers according to the invention may also contain one or more additional monomers, preferably cationic or nonionic monomers.

As compounds that are more particularly preferred according to the invention, mention may be made of the following terpolymers comprising at least:

a) one monomer of formula (XVI),

b) one monomer of formula (XIII) in which p=1, q=0, R₃ and R₄ denote, independently of each other, a hydrogen atom or a C₁-C₅ alkyl radical and R₅ denotes a C₉-C₂₄ alkyl radical, and

c) one monomer of formula (XIV) in which R₃ and R₄ denote, independently of each other, a hydrogen atom or a C₁-C₅ alkyl radical.

Even more preferentially, terpolymers comprising, by weight, 40% to 95% of monomer (a), 0.1% to 55% of monomer (c) and 0.25% to 50% of monomer (b) will be used.

Such polymers are described in patent application WO 00/68282, the content of which forms an integral part of the invention.

As cationic poly(vinyllactam) polymers according to the invention, vinylpyrrolidone/dimethylaminopropylmethacrylamide/dodecyldimethylmethacrylamidopropylammonium tosylate terpolymers, vinylpyrrolidone/dimethylaminopropylmethacrylamide/cocoylimethylmethacrylamidopropylammonium tosylate terpolymers and vinylpyrrolidone/dimethylaminopropylmethacrylamide/lauryldimethylmethacrylamidopropylammonium tosylate or chloride terpolymers are especially used.

The amphoteric associative polymers are preferably chosen from those comprising at least one noncyclic cationic unit. Even more particularly, the ones that are preferred are those prepared from or comprising 1 mol % to 20 mol %, preferably 1.5 mol % to 15 mol % and even more particularly 1.5 mol % to 6 mol % of fatty-chain monomer relative to the total number of moles of monomers.

The amphoteric associative polymers that are preferred according to the invention comprise or are prepared by copolymerizing:

1) at least one monomer of formula (XVII) or (XVIII):

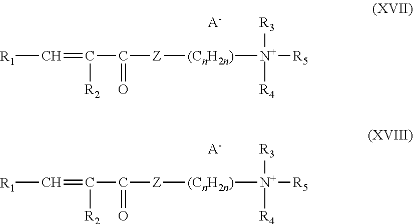

in which R₁ and R₂, which may be identical or different, represent a hydrogen atom or a methyl radical, R₃, R₄ and R₅, which may be identical or different, represent a linear or branched alkyl radical containing from 1 to 30 carbon atoms,

Z represents an NH group or an oxygen atom,

n is an integer from 2 to 5,

A⁻ is an anion derived from an organic or mineral acid, such as a methosulfate anion or a halide such as chloride or bromide;

2) at least one monomer of formula (XIX):

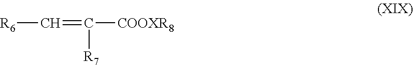

in which R₆ and R₇, which may be identical or different, represent a hydrogen atom or a methyl radical;

and

3) at least one monomer of formula (XX):

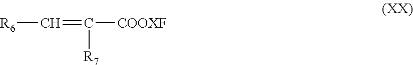

in which R₆ and R₇, which may be identical or different, represent a hydrogen atom or a methyl radical, X denotes an oxygen or nitrogen atom and R₈ denotes a linear or branched alkyl radical containing from 1 to 30 carbon atoms;

at least one of the monomers of formula (XVII), (XVIII) or (XX) comprising at least one fatty chain.

The monomers of formulae (XVII) and (XVIII) are preferably chosen from the group formed by:
dimethylaminoethyl methacrylate, dimethylaminoethyl acrylate,
diethylaminoethyl methacrylate, diethylaminoethyl acrylate,
dimethylaminopropyl methacrylate, dimethylaminopropyl acrylate,
dimethylaminopropylmethacrylamide, dimethylaminopropylacrylamide,

these monomers optionally being quaternized, for example with a C₁-C₄ alkyl halide or a C₁-C₄ dialkyl sulfate.

More particularly, the monomer of formula (XVII) is chosen from acrylamidopropyltrimethylammonium chloride and methacrylamidopropyltrimethylammonium chloride.

The monomers of formula (XIX) of the present invention are preferably chosen from the group formed by acrylic acid, methacrylic acid, crotonic acid and 2-methylcrotonic acid. More particularly, the monomer of formula (XIX) is acrylic acid.

The monomers of formula (XX) of the present invention are preferably chosen from the group formed by C₁₂-C₂₂ and more particularly C₁₆-C₁₈ alkyl acrylates or methacrylates.

The monomers constituting the fatty-chain amphoteric polymers of the invention are preferably already neutralized and/or quaternized.

The ratio of the number of cationic charges/anionic charges is preferably equal to about 1.

The amphoteric associative polymers of this family preferably comprise from 1 mol % to 10 mol % of the fatty-chain monomer (monomer of formula (XVII), (XVIII) or (XX)), and preferably from 1.5 mol % to 6 mol %.

The amphoteric associative polymers of this family may also contain other monomers such as nonionic monomers and in particular such as C₁-C₄ alkyl acrylates or methacrylates.

Amphoteric associative polymers according to the invention are described and prepared, for example, in patent application WO 98/44012.

Among the amphoteric associative polymers according to the invention, the ones that are preferred are acrylic acid/(meth)acrylamidopropyltrimethylammonium chloride/stearyl methacrylate terpolymers.

The associative polymers of nonionic type that may be used according to the invention are preferably chosen from:
(a) copolymers of vinylpyrrolidone and of fatty-chain hydrophobic monomers, of which examples that may be mentioned include:
   the products Antaron V216® and Ganex V216® (vinylpyrrolidone/hexadecene copolymer) sold by the company ISP.
   the products Antaron V220® and Ganex V220® (vinylpyrrolidone/eicosene copolymer) sold by the company ISP.
(b) copolymers of C₁-C₆ alkyl methacrylates or acrylates and of amphiphilic monomers comprising at least one fatty chain, such as, for example, the oxyethylenated methyl acrylate/stearyl acrylate copolymer sold by the company Goldschmidt under the name Antil 208®.
(c) copolymers of hydrophilic methacrylates or acrylates and of hydrophobic monomers comprising at least one fatty chain, for instance the polyethylene glycol methacrylate/lauryl methacrylate copolymer.
(d) polyurethane polyethers comprising in their chain both hydrophilic blocks usually of polyoxyethylenated nature and hydrophobic blocks, which may be aliphatic sequences alone and/or cycloaliphatic and/or aromatic sequences.
(e) polymers with an aminoplast ether backbone containing at least one fatty chain, such as the Pure Thix® compounds sold by the company Sud-Chemie.
(f) celluloses or derivatives thereof, modified with groups comprising at least one fatty chain, such as alkyl, arylalkyl or alkylaryl groups or mixtures thereof in which the alkyl groups are of C₈, and in particular:
   nonionic alkylhydroxyethylcelluloses such as the products Natrosol Plus Grade 330 CS and Polysurf 67 (C₁₆ alkyl) sold by the company Aqualon;
   nonionic nonoxynylhydroxyethylcelluloses such as the product Amercell HM-1500 sold by the company Amerchol;
   nonionic alkylcelluloses such as the product Bermocoll EHM 100 sold by the company Berol Nobel;
(g) associative guar derivatives, for instance hydroxypropyl guars modified with a fatty chain, such as the product Esaflor HM 22 (modified with a C₂₂ alkyl chain) sold by the company Lamberti; the product Miracare XC 95-3 (modified with a C₁₄ alkyl chain) and the product RE 205-146 (modified with a C₂₀ alkyl chain) sold by Rhodia Chimie.

Preferably, the polyurethane polyethers comprise at least two hydrocarbon-based lipophilic chains containing from 6 to 30 carbon atoms, separated by a hydrophilic block, the hydrocarbon-based chains possibly being pendent chains or chains at the end of the hydrophilic block. In particular, it is possible for one or more pendent chains to be included. In addition, the polymer may comprise a hydrocarbon-based chain at one end or at both ends of a hydrophilic block.

The polyurethane polyethers may be multiblock, in particular in triblock form. The hydrophobic blocks may be at each end of the chain (for example: triblock copolymer containing a hydrophilic central block) or distributed both at the ends and in the chain (for example multiblock copolymer). These same polymers may also be graft polymers or star polymers.

The nonionic fatty-chain polyurethane polyethers may be triblock copolymers in which the hydrophilic block is a polyoxyethylenated chain comprising from 50 to 1000 oxyethylene groups. The nonionic polyurethane polyethers comprise a urethane bond between the hydrophilic blocks, whence arises the name.

By extension, also included among the nonionic fatty-chain polyurethane polyethers are those in which the hydrophilic blocks are linked to the lipophilic blocks via other chemical bonds.

As examples of nonionic fatty-chain polyurethane polyethers that may be used in the invention, it is also possible to use Rheolate 205® containing a urea function, sold by the company Rheox, or Rheolate® 208, 204 or 212, and also Acrysol RM 184®.

Mention may also be made of the product Elfacos T210® containing a C₁₂-C₁₄ alkyl chain, and the product Elfacos T212® containing a C₁₈ alkyl chain, from Akzo.

The product DW 1206B® from Röhm & Haas containing a C₂₀ alkyl chain and a urethane bond, sold at a solids content of 20% in water, may also be used.

It is also possible to use solutions or dispersions of these polymers, especially in water or in aqueous-alcoholic medium. Examples of such polymers that may be mentioned are Rheolate® 255, Rheolate® 278 and Rheolate® 244 sold by the company Rheox. The products DW 1206F and DW 1206J sold by the company Röhm & Haas may also be used.

The polyurethane polyethers that may be used according to the invention are in particular those described in the article by G. Formum, J. Bakke and Fk. Hansen—Colloid Polym. Sci. 271, 380.389 (1993).

It is even more particularly preferred to use a polyurethane polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane polyethers are sold especially by the company Röhm & Haas under the names Aculyn 46® and Aculyn 44® [Aculyn 46® is a polycondensate of polyethylene glycol containing 150 or 180 mol of ethylene oxide, of stearyl alcohol and of methylenebis(4-cyclohexyl isocyanate) (SMDI), at 15% by weight in a matrix of maltodextrin (4%) and water (81%); Aculyn 44® is a polycondensate of polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)].

Even more preferentially, the thickening polymer(s) of the invention are chosen from associative or nonassociative polymers bearing sugar units, associative or nonassociative acrylic or methacrylic anionic polymers, and associative or nonassociative polyurethanes.

The preferred conditioning polymers are preferably chosen from cationic or amphoteric, preferably cationic, conditioning polymers.

Among the conditioning polymers that may be used, it is preferred to use quaternary cellulose ether derivatives such as the products sold under the name JR 400 or Quatrisoft LM 200 by the company Amerchol, cationic cyclopolymers, in particular diallyldimethylammonium chloride homopolymers and copolymers, such as those sold under the names Merquat® 100, Merquat® 550 and Merquat® S by the company Nalco, cationic modified guar gums such as Jaguar C13S, quaternary polymers of vinylpyrrolidone and of vinylimidazole, and homopolymers of ethyltrimethylammonium methacrylate salts (Salcare SC95 or 96), and mixtures thereof.

Most particularly, the nonsilicone polymers of the invention are chosen from associative or nonassociative polymers bearing sugar units, and better still from polysaccharides of hydroxyethylcellulose and hydroxypropyl guar type.

The nonsilicone polymer(s) may especially be present in an amount ranging from 0.01% to 20% by weight, preferably from 0.1% to 10% by weight and better still from 0.2% to 5% by weight, relative to the total weight of the composition.

The weight ratio of the amount of pumice particles to the amount of nonsilicone polymers preferably ranges from 1 to 200, even more preferentially from 3 to 100 and better still from 5 to 50.

The haircare composition according to the invention may also comprise at least one silicone containing quaternary ammonium groups.

In accordance with the invention, the term “silicone containing quaternary ammonium groups” means any silicone comprising one or more quaternary ammonium groups. These quaternary ammonium groups may be attached in the alpha or omega position or in the form of side groups. They may be attached directly to the polysiloxane backbone or may be borne by hydrocarbon-based chains.

According to the invention, the term “silicone” means, in accordance with what is generally accepted, any polymer having a structure based on an alternation of silicon and oxygen atoms, linked together via bonds known as siloxane bonds (—Si—O—Si—), and also characterized by the existence of silicon-carbon bonds. These silicones, or polysiloxanes, are generally obtained by polycondensation of suitably functionalized silanes. The hydrocarbon-based radicals most commonly borne by the silicon atoms are lower alkyl radicals, in particular methyl, fluoroalkyl radicals, and aryl radicals and in particular phenyl.

The silicones containing quaternary ammonium groups of the present invention are chosen, for example, from the compounds corresponding to the following general formulae:

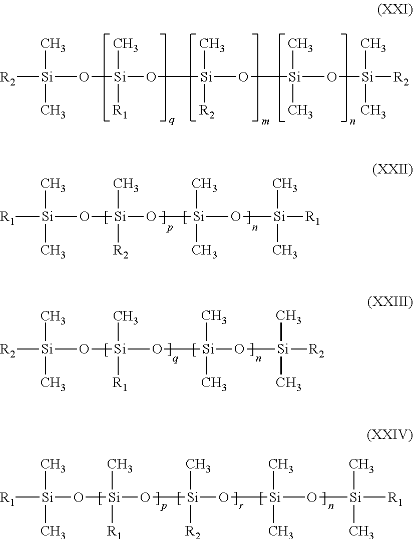

in which:
R₁, which may be identical or different, represents a linear or branched C₁-C₃₀ alkyl group or a phenyl group;
R₂, which may be identical or different, represents —C_{c}H_{2c}—O—(C₂H₄O)ₐ—(C₃H₆O)_{b}—(PO₃H)_{d}—R₅ or —C_{c}H_{2c}—O—(C₄H₈O)ₐ—(PO₃H)_{d}—R₅;

R₅, which may be identical or different, is chosen from the groups of the following formula:

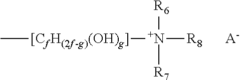

the radicals R₈ independently represent a linear or branched C₁-C₂₂ alkyl or C₂-C₂₂ alkenyl radical, optionally bearing one or more OH groups, or represent a group CₕH₂ₕZCOR₉; and
R₆, R₇ and R₉, which may be identical or different, represent linear or branched C₁-C₂₂ alkyl or C₂-C₂₂ alkenyl radicals, optionally bearing one or more OH groups, or R₇ may form with part of R₈ a heterocycle (ring containing at least one heteroatom, for instance N, O or P), the heterocycle especially being an imidazoline.

Preferably, R₆ and R₇ denote a C₁-C₆ alkyl radical and more particularly methyl, R₉ preferably denotes a radical chosen from C₈-C₁₈ alkyl and C₈-C₁₈ alkenyl and especially a cocoyl radical.
m ranges from 0 to 20;
n ranges from 0 to 500;
p ranges from 1 to 50;
q ranges from 0 to 20;
r ranges from 1 to 20;
a ranges from 0 to 50;
b ranges from 0 to 50;
c ranges from 0 to 4;
d denotes 0 or 1;
f ranges from 0 to 4;
g ranges from 0 to 2, and is preferably equal to 1; and
h ranges from 1 to 4, and is preferably equal to 3;

Z represents an oxygen atom or NH;

A⁻ represents a monovalent organic or inorganic anion such as a halide (e.g. chloride, bromide), a sulfate or a carboxylate (e.g. acetate, lactate, citrate).

Silicones containing quaternary ammonium groups of formula (XXII) or (XXIII) are preferably used.

It is preferred to use silicones containing quaternary ammonium groups corresponding to the general formula (XXIII) as defined above, and more particularly those corresponding to the general formula (XXIII) in which at least one, and preferably all, of the following conditions are met:
c is equal to 0;
d denotes 0;
a is equal to zero;
b is equal to 1;
n ranges from 0 to 100;
q is equal to 0;
f=3;
g=1;
R₆ and R₇ denote a methyl group; and
R₈ denotes a C₁₀-C₂₂ alkyl radical.

Among the silicones of the invention, examples that may be mentioned include those sold by the company Goldschmidt under the names Abil Quat 3272, Abil B 9905, Abil Quat 3474 and Abil K 3270, by the company Lipo France under the names Silquat Q-100, Silquat Q-200 WS, Silquat AX, Silquat AC, Silquat AD and Silquat AM, all manufactured by the company Siltech, by the company OSI under the name Magnasoft Exhaust and Silsoft C-880, and by the company UCIB under the names Pecosil 14-PQ and Pecosil 36-PQ (manufactured by Phoenix Chemical). These silicones are especially described in patents EP 0 530 974, DE 3 719 086, DE 3 705 121, EP 0 617 607 and EP 0 714 654.

According to one embodiment, the silicone containing quaternary ammonium groups is of formula (XXIII). Even more preferentially, the silicone containing quaternary ammonium groups is the compound referenced in the CTFA (INCI name) under the name Quaternium-80.

The silicones containing quaternary ammonium groups used in accordance with the invention may be in the form of aqueous solutions, or optionally in the form of dispersions or emulsions in water.

The haircare composition may comprise silicones containing quaternary ammonium groups in an amount ranging from 0.1% to 20% by weight, preferably from 0.2% to 10% by weight and better still from 0.3% to 5% by weight, relative to the total weight of the composition.

The weight ratio of the amount of pumice particles to the amount of silicones containing quaternary ammonium groups preferably ranges from 1 to 250, even more preferentially from 5 to 200 and better still from 10 to 100.

Other Components of the Haircare Composition

Besides the pumice particles, the cationic surfactant(s) comprising at least one ester function and the nonsilicone polymer(s) in accordance with the invention, the haircare composition may comprise one or more additives.

The haircare composition may thus comprise one or more additional surfactants such as anionic, amphoteric, zwitterionic or nonionic surfactants.

The additional surfactant(s) are preferably chosen from nonionic surfactants.

Nonionic surfactants are compounds that are well known per se (see especially in this regard the “Handbook of Surfactants” by M. R. Porter, published by Blackie & Son (Glasgow and London), 1991, pp. 116-178). Thus, they may be chosen especially from (nonlimiting list) polyethoxylated or polypropoxylated alcohols, alpha-diols and alkylphenols, containing a fatty chain comprising, for example, 8 to 18 carbon atoms, the number of ethylene oxide or propylene oxide groups possibly ranging especially from 2 to 50. Mention may also be made of ethylene oxide and propylene oxide copolymers, condensates of ethylene oxide and of propylene oxide with fatty alcohols; polyethoxylated fatty amides preferably having from 2 to 30 ethylene oxide units, polyglycerolated fatty amides containing on average 1 to 5, and in particular 1.5 to 4, glycerol groups, ethoxylated fatty acid esters of sorbitan containing from 2 to 30 ethylene oxide units, fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

The nonionic surfactant(s) may be present in the haircare composition in concentrations ranging from 0.1% to 25% by weight and preferably from 1% to 20% by weight, relative to the total weight of the composition.

As regards the amphoteric or zwitterionic surfactants, mention may be made, without the intention of being limited thereto, of aliphatic secondary or tertiary amine derivatives, in which the aliphatic radical is a linear or branched chain comprising 8 to 18 carbon atoms and containing at least one water-solubilizing anionic group (for example carboxylate, sulfonate, sulfate, phosphate or phosphonate); mention may also be made of (C₈-C₂₀)alkylbetaines, sulfobetaines, (C₈-C₂₀)alkylamido(C₁-C₆)alkylbetaines or (C₈-C₂₀)alkylamido(C₁-C₆)alkylsulfobetaines.

Among the amine derivatives, mention may be made of the products sold under the name Miranol, as described in U.S. Pat. No. 2,528,378 and U.S. Pat. No. 2,781,354 and classified in the CTFA dictionary, 3rd edition, 1982, under the names Amphocarboxyglycinate and Amphocarboxypropionate, having the respective structures:

R₂—CONHCH₂CH₂—N(R₃)(R₄)(CH₂COO⁻)

in which: R₂ denotes an alkyl radical of an acid R₂—COOH present in hydrolyzed coconut oil, a heptyl, nonyl or undecyl radical, R₃ denotes a β-hydroxyethyl group and R₄ a carboxymethyl group; and

R₂′—CONHCH₂CH₂—N(B)(C)

in which:

B represents —CH₂CH₂OX′, C represents —(CH₂)_{z}—Y′, with z=1 or 2,

X′ denotes the —CH₂CH₂—COOH group or a hydrogen atom,

Y′ represents —COOH or the —CH₂CHOH—SO₃H radical, and

R₂′ denotes an alkyl radical of an acid R₉—COOH present in coconut oil or in hydrolyzed linseed oil, an alkyl radical, especially of C₇, C₉, C₁₁ or C₁₃, a C₁₇ alkyl radical and its iso form, or an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid and disodium cocoamphocarboxyl ethyl hydroxypropyl sulfonate.

An example that may be mentioned is the cocoamphodiacetate sold under the trade name Miranol® C2M Concentrate by the company Rhodia Chimie.

As regards the anionic surfactants, mention may be made in a nonlimiting manner of the salts (in particular of sodium, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; (C₆-C₂₄)alkyl sulfosuccinates, (C₆-C₂₄)alkyl ether sulfosuccinates, (C₆-C₂₄)allylamide sulfosuccinates; (C₆-C₂₄)alkyl sulfoacetates; (C₆-C₂₄)acyl sarcosinates and (C₆-C₂₄)acyl glutamates. It is also possible to use (C₆-C₂₄)alkylpolyglycoside carboxylic esters such as alkylglucoside citrates, alkylpolyglycoside tartrates and alkylpolyglycoside sulfosuccinates, alkylsulfosuccinamates; acyl isethionates and N-acyl taurates, the alkyl or acyl radical of all of these different compounds preferably containing from 12 to 20 carbon atoms and the aryl radical preferably denoting a phenyl or benzyl group.

Among the anionic surfactants that may also be used, mention may also be made of fatty acid salts such as oleic, ricinoleic, palmitic and stearic acid salts, coconut oil acid or hydrogenated coconut oil acid; acyl lactylates in which the acyl radical contains 8 to 20 carbon atoms. It is also possible to use alkyl-D-galactoside uronic acids and salts thereof, polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and salts thereof, in particular those containing from 2 to 50 alkylene oxide and in particular ethylene oxide groups, and mixtures thereof.

The anionic surfactants that may be present are preferably mild anionic surfactants.

As regards the mild anionic surfactants, mention may be made especially of the following compounds and salts thereof, and also mixtures thereof:
polyoxyalkylenated alkyl ether carboxylic acids;
polyoxyalkylenated alkylaryl ether carboxylic acids;
polyoxyalkylenated alkylamido ether carboxylic acids, in particular those comprising 2 to 50 ethylene oxide groups;
alkyl-D-galactoside uronic acids;
acylsarcosinates, acylglutamates; and
alkylpolyglycoside carboxylic esters.

It is most particularly possible to use polyoxyalkylenated alkyl ether carboxylic acids, for instance lauryl ether carboxylic acid (4.5 EO) sold, for example, under the name Akypo RLM 45 CA from Kao.

If such anionic or amphoteric surfactants are present, then their content ranges from 0.1% to 20% by weight relative to the total weight of the haircare composition, and more particularly from 1% to 10% by weight relative to the total weight of the composition.

Preferably, the haircare composition does not contain any anionic detergent surfactant of sulfate type (alkyl sulfate or alkyl ether sulfate, alkylamido ether sulfate). If it does contain any, its content is such that the weight ratio: anionic detergent surfactant of alkyl sulfate or alkyl ether sulfate type/sum of the other noncationic surfactants is preferably less than or equal to 1, more particularly less than or equal to 0.75 and even more preferentially less than or equal to 0.5.

The haircare composition may moreover comprise additives that are conventional in the field, for instance those chosen from the nonexhaustive list such as reducing agents, oxidizing agents, sequestrants, softeners, antifoams, moisturizers, emollients, basifying agents, plasticizers, sunscreens, direct dyes or oxidation dyes, fragrances, peptizers, preserving agents, vitamins, antidandruff agents, antiseborrheic agents, hair-loss counteractants, and nonpolymeric thickeners such as fatty amides, fatty ethers, fatty alcohols, silicas, clays, etc.

The adjuvants mentioned above are generally present in an amount, for each of them, of between 0.01% and 20% by weight, relative to the weight of the composition.

The haircare composition may comprise one or more additional nonquaternary nonpolymeric or silicone conditioning agents.

When the haircare composition contains at least one additional conditioning agent, this agent may be chosen from synthetic oils such as poly-α-olefins, fluoro oils, fluoro waxes, fluoro gums, carboxylic acid esters, cationic surfactants other than those required according to the invention, nonquaternary silicones, mineral, plant or animal oils, ceramides and pseudoceramides, and mixtures thereof.

Among the cationic surfactants other than those required according to the invention, mention may be made in particular (nonlimiting list) of optionally polyoxyalkylenated primary, secondary or tertiary fatty amine salts; imidazoline derivatives; quaternary ammonium salts and in particular trialkylammonium salts comprising at least one fatty chain containing from 10 to 30 carbon atoms and especially cetyltrimethylammonium or behenyltrimethylammonium salts.

The content of additional nonquaternary nonpolymeric or silicone conditioning agents in the haircare composition may range from 0.001% to 10% by weight, preferably from 0.005% to 5% by weight and even more preferentially from 0.01% to 3% by weight relative to the total weight of the final composition.

The aqueous medium that is acceptable for the hair may comprise water or a mixture of water and of one or more cosmetically acceptable organic solvents.

The content of water in the composition is preferably greater than or equal to 50% by weight relative to the total weight of the composition.

Examples of organic solvents that may be mentioned include linear or branched and preferably saturated monoalcohols comprising 2 to 10 carbon atoms, such as ethyl alcohol or isopropyl alcohol; aromatic alcohols such as benzyl alcohol and phenylethyl alcohol; polyols or polyol ethers, for instance ethylene glycol monomethyl, monoethyl and monobutyl ethers, propylene glycol or ethers thereof, for instance propylene glycol monomethyl ether, butylene glycol or dipropylene glycol, hexylene glycol (2-methyl-2,4-pentanediol), neopentyl glycol and 3-methyl-1,5-pentanediol; and also diethylene glycol alkyl ethers, especially of C₁-C₄, for instance diethylene glycol monoethyl ether or monobutyl ether, alone or as a mixture.

The organic solvent(s) may be present in proportions, for example, of between 1% and 40% by weight approximately relative to the total weight of the composition according to the invention, and even more preferentially between 5% and 30% by weight approximately.

Galenical Form

The haircare composition according to the invention may be in various galenical forms, such as a lotion, a shampoo, a gel, a cream or a wax. The haircare composition may be conditioned in any type of container with or without an applicator. The container may contain a roller or a member making it possible especially to homogenize the haircare composition before its application to the hair. The container containing the haircare composition may have a capacity of greater than or equal to 15 ml, especially greater than or equal to 50 ml, or even 100 ml, especially greater than or equal to 150 ml, for example between 15 ml and 500 ml.

The haircare composition may impregnate a wipe.

The haircare composition may be introduced into a stream of carrier fluid, and be sprayed onto the hair.

In one particular embodiment, the haircare composition may be contained in a reservoir of a comb or a brush having, for example, orifices at the base of the teeth or bristles, for delivering the product and applying it to the hair.

Treatment Process

The invention also relates to a hair treatment process that consists in applying to the hair at least one haircare composition as defined previously, and then optionally in rinsing it out.

The treatment, for example abrasion, of the hair, may take place immediately after application of the haircare composition to the hair, for example within a period of less than one hour, especially less than 30 minutes, or even 10 minutes, after application of the haircare composition to the hair. Rinsing of the hair, when it is performed, may take place rapidly after treatment, for example abrasion, of the hair. The time between the application of the composition and rinsing may be, for example, less than 4 hours, especially less than 1 hour, for example less than 20 minutes.

The step of treatment, for example abrasion, of the hair may be performed by rubbing the hair with the bare hands or by placing at least one surface between the hands and the hair charged with haircare composition, for example a wipe or towel, a glove or the like. The treatment may be performed other than by using a comb or a brush.

The treatment, for example abrasion, of the hair may be performed, for example, using at least one surface placed in motion, in vibration or in rotation, especially by a mechanized system.

The treatment using the haircare composition may be performed, for example, by hand, for example lock by lock, in two movements: a first “tangential,” movement, i.e. substantially along the hair, for example from the root to the end, and a second “shear” motion, i.e. substantially orthogonal to the hair, in a transverse direction thereto. The two movements may be repeated several times, for example at least five times, for example ten times. As a variant, only one of the abovementioned movements may be performed and optionally repeated several times.

The duration of treatment, for example abrasion, using the composition in accordance with the invention may depend, for example, on the desired intensity of the abrasion and on the condition of the hair.

The process according to the invention may comprise two treatment steps, for example abrasion, using two haircare compositions applied successively, each comprising pumice particles in accordance with the invention, the mean volume diameters or the hardness of the pumice particles in accordance with the invention of the two compositions being different.

A single bottle may contain the two haircare compositions conditioned separately, or two different bottles may each contain a haircare composition.

Another conditioning mode may consist of a bottle containing a base haircare composition free of pumice particles in accordance with the invention, which are conditioned separately, for example in at least two separate compartments, as a function of their particle size or their hardness. In the latter case, the user selects before the treatment, for example abrasion, the pumice particles in accordance with the invention to be mixed with the base composition to form the first haircare composition, the mixing being performed by the user or in the bottle. After the first treatment, for example the first abrasion, the user may perform a second treatment, for example a second abrasion, this time mixing other pumice particles in accordance with the invention with the base haircare composition to form the second haircare composition. Rinsing may be performed between and/or after the two treatments.

The treatment process may be performed after a step of characterization of the hair, for example by means of a visual examination with the naked eye or under a magnifying device or by instrumental means, for example by recording the sound produced by the movement of a comb through the hair using a sonometer or by optically determining the gloss of the hair. The characterization of the hair may also involve a chemical reagent applied onto a sample of hair.

The process according to the invention may include the step consisting in combing and/or rinsing the hair after application of the haircare composition. The rinsing may be performed with water.

The process according to the invention may also include a step that consists in heating the hair before or after placing in contact with the haircare composition, for example to a temperature of between 40° C. and 250° C. and especially between 60° C., and 220° C. The hair may, for example, be heated after the treatment, so as to shape it, for example by performing blow-drying. The heating of the hair may be performed, for example, using an iron, a liquid water/steam mixture or using a heating hood.

The hair may be totally or partially dried.

Application of a Treatment Product

The process according to the invention may also include the step consisting in applying, for example before or after treatment using the haircare composition according to the invention, another treatment product to the hair. The treatment product may be, for example, a cosmetic product, especially a conditioner, a permanent-waving product, a relaxer or a product for dyeing or bleaching the hair.

The treatment product may be chosen, for example, from the following products, this list not being limiting:
products for modifying the mechanical properties of the hair, especially comprising a reducing agent, such as thioglycolic acid and derivatives thereof, cysteine, sulfite, sodium hydroxide, guanidine carbonate, trihydroxymethylphosphine, or an oxidizing agent, such as H₂O₂ or persulfate;
emollients or penetrants, comprising, for example, a solvent, a glycol, a plasticizer or a cationic, anionic or amphoteric surfactant;
products that modify the surface properties of the hair, especially comprising a silicone, a reactive aminosilicone, an adhesive polymer, a nonsilicone lubricant comprising fatty substances chosen from plant oils, mineral oils, synthetic oils, and waxes, especially fatty alcohols or fatty esters;
products for restructuring the interior of the hair, comprising, for example, an ionene, a protein, a hydroxy acid or a reactive compound, especially a formaldehyde generator, a silane; and
direct dyes or oxidation dyes.

The treatment product may be applied before the treatment, for example of abrasion type, and may contribute toward protecting the hair during said treatment, in order especially to avoid excessive abrasion.

The treatment product is preferably applied after application of the composition in accordance with the invention.

Thus, according to one embodiment, the process in accordance with the invention may also include the step that consists in subjecting the hair to a post-treatment, after treatment using a haircare composition as defined previously, the post-treatment being chosen from the application of a conditioner, a permanent-waving product, a relaxer, and a product for dyeing or bleaching the hair.

Treatment Kits

A subject of the invention is also, independently or in combination with the foregoing, a hair treatment kit comprising:
a haircare composition comprising at least pumice particles, one or more cationic surfactants comprising at least one ester function and one or more nonsilicone polymers, and
packaging comprising instructions for the use of the haircare composition on the hair, for example in order to perform abrasion of the hair.

The haircare composition is as described hereinabove.

The kit may also comprise a hair post-treatment composition. The post-treatment composition may be chosen from a conditioner, a permanent-waving product, a relaxer, and a product for dyeing or bleaching the hair. The post-treatment product may be chosen, for example, from those described above.

### EXAMPLE

(the proportions are on a weight basis relative to the total weight of the composition)

### Comparative Example 1

### Demonstration of the Smoothing Power

The following treatment compositions were produced:

| Composition | A | B |
|---|---|---|
| Caprylyl glycol | 0.5 | 0.5 |
| Cetrimonium chloride | 0.5 | 0.5 |
| Pumice (Pierre Ponce 0½ D from Eyraud) | 18 | 0 |
| Quaternium-80 (Abil Quat 3272 from | 0.2 | 0.2 |
| Goldschmidt) | | |
| Glyceryl stearate | 1.2 | 1.2 |
| Hydroxypropyl guar (Jaguar HP 105 from | 0.1 | 0.1 |
| Rhodia) | | |
| Glycerol | 2 | 2 |
| Propylene glycol | 0.5 | 0.5 |
| Cetearyl alcohol | 4 | 4 |
| Hydroxyethylcellulose (Natrosol 250 HHR | 0.8 | 0.8 |
| from Aqualon) | | |
| Preserving agents | 0.2 | 0.2 |
| Dipalmitoylethylhydroxyethylammonium | 4.3 | 4.3 |
| methosulfate/Cetearyl alcohol (30/70 by | | |
| weight) (Dehyquart F30 from Cognis) | | |
| Water | qs 100 | qs 100 |

The study is performed on a panel of 20 women: 10 women with long natural hair and 10 women with long sensitized hair.

10 to 15 g of each of these treatments are applied per half-head.

The application is performed lock by lock in two movements: a first “tangential” movement and a second “shear” movement. The two movements are repeated ten times. The hair is combed, then rinsed and finally dried by blow-drying.

Sensory Analysis

The results show that for 7 out of 10 women with natural hair and for 9 out of 10 women with sensitized hair, the side treated via the process of the invention with composition A is smoother, more supple and more uniform than the side treated with composition B. The invention makes it possible to reduce the mass and to control the volume of the hair, and to improve the manageability of the head of hair. The effects are all the more noteworthy when the hair is sensitized and thick.

After five shampoo washes, the smoothness of the hair is conserved, as is the volume control, irrespective of the degree of sensitization of the hair (natural to very sensitized).

### Example 2

The following composition was prepared:

| | |
|---|---|
| Caprylyl glycol | 0.5 |
| Cetrimonium chloride | 0.5 |
| Pumice (0-D decontaminated pumice from Eyraud, | 25 |
| less than 125 μm in size) | |
| Quaternium-80 | 0.2 |
| (Abil Quat 3272 from Goldschmidt) | |
| Glyceryl stearate | 1.2 |
| Hydroxypropyl guar (Jaguar HP 105 from Rhodia) | 0.1 |
| Glycerol | 2 |
| Propylene glycol | 0.5 |
| Cetearyl alcohol | 1 |
| Hydroxyethylcellulose (Natrosol 250 HHR from Aqualon) | 0.8 |
| Preserving agents | 0.2 |
| Dipalmitoylethylhydroxyethylammonium | 4.3 |
| methosulfate/Cetearyl alcohol (30/70 by weight) | |
| (Dehyquart F30 from Cognis) | |
| Water | qs 100 |

### Example 3

The following composition was prepared:

| | |
|---|---|
| Caprylyl glycol | 0.1 |
| Cetrimonium chloride | 0.5 |
| Pumice (Pierre Ponce 0½ D from Eyraud) | 15 |
| Glyceryl stearate | 1.2 |
| Hydroxypropyl guar (Jaguar HP 105 from Rhodia) | 0.1 |
| Glycerol | 2 |
| Propylene glycol | 0.5 |
| Cetearyl alcohol | 1 |
| Hydroxyethylcellulose (Natrosol 250 HHR from Aqualon) | 0.8 |
| Preserving agents | 0.2 |
| Dipalmitoylethylhydroxyethylammonium | 4.3 |
| methosulfate/Cetearyl alcohol (30/70 by weight) | |
| (Dehyquart F30 from Cognis) | |
| Water | qs 100 |

The invention is not limited to the described examples.

The term “comprising one” should be understood as being synonymous with “comprising at least one”, and “between” or “ranging from” are understood as including the limits, unless otherwise specified.

1. A hair care composition comprising at least pumice particles, one or more cationic surfactants comprising at least one ester function, and one or more nonsilicone polymers.

2. The composition of claim 1, wherein the pumice particles have a mean volume diameter of less than or equal to 500 μm.

3. The composition of claim 1, wherein the pumice particles are present in a content ranging from 0.1% to 35% by weight, relative to the total weight of the composition.

4. The composition of claim 1, wherein the pumice particles have a hardness ranging from 3 to 10 Moh.

5. The composition of claim 1, wherein said cationic surfactant comprising at least one ester function is chosen from quaternary ammonium salts containing at least one ester function of formula (I) below: 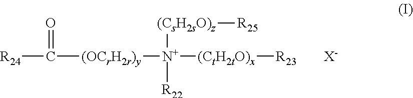
wherein
R₂₂ is chosen from C₁-C₆ alkyl radicals and C₁-C₆ hydroxyalkyl or dihydroxyalkyl radicals;
R₂₃ is selected from:
the radical 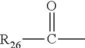
linear or branched, saturated or unsaturated C₁-C₂₂ hydrocarbon-based radicals R₂₇, and
a hydrogen atom,
R₂₅ is selected from:
the radical 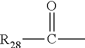
and
linear or branched, saturated or unsaturated C₁-C₆ hydrocarbon-based radicals R₂₉, and
a hydrogen atom,
R₂₄, R₂₆ and R₂₈, which may be identical or different, are selected from linear or branched, saturated or unsaturated C₇-C₂₁ hydrocarbon-based radicals;
r, s and t are integers ranging from 2 to 6;
y is an integer ranging from 1 to 10;
x and z are integers ranging from 0 to 10; and
X⁻ is a simple or complex, organic or inorganic anion;
with the proviso that the sum x+y+z is from 1 to 15, that when x is 0 then R₂₃ denotes R₂₇, and that when z is 0 then R₂₅ denotes R₂₉.

6. The composition of claim 5, wherein:
R₂₂ denotes a methyl or ethyl radical,
x and y are equal to 1,
z is equal to 0 or 1,
r, s and t are equal to 2,
R₂₃ is selected from:
the radical 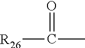
methyl, ethyl or C₁₄-C₂₂ hydrocarbon-based radicals,
a hydrogen atom;
R₂₅ is selected from:
the radical 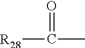
and
a hydrogen atom;
R₂₄, R₂₆ and R₂₈ are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

7. The composition of claim 1, wherein the cationic surfactant comprising at least one ester function is a diacyloxydialkylhydroxyalkylammonium salt, a diacyloxytrialkylammonium salt, dipalmitoylethylhydroxyethylammonium methosulfate, dicetearoylethylhydroxyethylmethylammonium methosulfate, distearoylethyldimethylammonium chloride or distearoyldiethylmethylammonium chloride.

8. The composition of claim 1, wherein the cationic surfactant(s) comprising at least one ester function are present in a content ranging from 0.1% to 15% by weight, relative to the total weight of the composition.

9. The composition of claim 1, wherein the nonsilicone polymer(s) are chosen from nonionic, anionic, cationic or amphoteric thickening, fixing or conditioning polymers.

10. The composition of claim 9, wherein the thickening polymer(s) are chosen from associative or nonassociative polymers bearing sugar units, associative or nonassociative acrylic or methacrylic anionic polymers, and associative or nonassociative polyurethanes.

11. The composition of claim 9, wherein the conditioning polymer(s) are chosen from quaternary cellulose ether derivatives, cationic cyclopolymers, dimethyldiallylammonium chloride homopolymers or copolymers, cationic modified guar gums, quaternary polymers of vinylpyrrolidone and of vinylimidazole, and homopolymers of ethyltrimethylammonium methacrylate salts, and mixtures thereof.

12. The composition of claim 1, wherein the nonsilicone polymer(s) are present in a content ranging from 0.01% to 20% by weight, relative to the total weight of the composition.

13. The composition of claim 1, further comprising at least one silicone-containing quaternary ammonium groups.

14. The composition of claim 13, wherein said silicone is Quaternium-80.

15. A process for treating hair, said process comprising the steps of applying to the hair at least one hair-care composition comprising at least pumice particles, one or more cationic surfactants comprising at least one ester function, and one or more nonsilicone polymers; and rinsing the at least one hair-care composition out.

16. The process of claim 15, further comprising the step of applying to the hair a conditioner, a permanent-waving product, a relaxer, or a product for dyeing or bleaching the hair.

17-19. (canceled)

20. The composition of claim 1, wherein the pumice particles have a mean volume of between 100 and 300 μm.

21. The composition of claim 1, wherein the pumice particles are present in a content ranging from 18% to 20% by weight, relative to the total weight of the composition.

22. The composition of claim 1, wherein the pumice particles have a hardness ranging from 5 to 5.5 on the Moh scale

23. The composition of claim 1, wherein the cationic surfactant(s) comprising at least one ester function are present in a content ranging from 0.1% to 5% by weight, relative to the total weight of the composition.
